(19) United States
(12) Patent Application Publication
(10) Pub. No.: US 2021/0290442 A1
(43) Pub. Date: Sep. 23, 2021

(54) ELASTIC BANDAGE WITH ENGAGEMENT MEANS

(71) Applicants: Trident Holding IVS, Helsingør (DK); AGLP Holding ApS, Holstebro (DK)

(72) Inventors: Allan Pederson, Holstebro (DK); Kristian Eggeling, Helsingor (DK)

(21) Appl. No.: 16/624,855

(22) PCT Filed: Mar. 8, 2018
(86) PCT No.: PCT/EP2018/055767

§ 371 Date: Dec. 19, 2019

(30) Foreign Application Priority Data
Jun. 29, 2017 (DK) PA 2017 00393

(51) Int. Cl. A61F 13/00

(52) U.S. Cl. CPC A61F 13/00021; A61F 2013/00119; A61F 2013/00174; A61F 2013/00106

(57) ABSTRACT

An elastic bandage includes an elastic, elongated material having an elongated length substantially longer than a width of the material. The elastic bandage further includes an engagement means joined to a first side surface of the elongated material and being sized and adapted for the elongated material to be pulled around.

### TECHNICAL FIELD OF THE INVENTION

The present invention relates to the field of elastic bandages.

### BACKGROUND OF THE INVENTION

In both military and non-military emergency medical situations, it is often necessary to temporarily close a wound to prevent bleeding and to avoid contaminating the wound. In many situations, it is impractical to suture or staple a wound closed, which will often take five to ten minutes.

While utilization of strap style tourniquets has been widely accepted for field care, these devices do not address major bleeding in the axillary area (arm pit) and inguinal area (groin area), where larger blood vessels run. Therefore, haemorrhage control is the leading cause of death in the pre-hospital environment.

### OBJECT OF THE INVENTION

The objective of the present invention is to provide a bandage for military and non-military emergency medical situations that can be used to temporarily close a wound in the axillary area and inguinal area.

### DESCRIPTION OF THE INVENTION

One aspect of the present invention relates to an elastic bandage, comprising: an elastic, elongated material having an elongated length substantially longer than a width of the material; wherein the elastic bandage further comprises an engagement means joined to a first side surface of the elongated material, and being sized and adapted for the elongated material to be pulled around.

Another aspect of the present invention relates to an elastic bandage, comprising:

an elastic, elongated material having an elongated length substantially longer than a width of the material; wherein the elastic bandage further comprises an engagement means joined to a first side surface of the elongated material, and being sized and adapted for the elongated material to be pulled around during use of the elastic bandage.

Yet another aspect relates to an elastic bandage, comprising:

an elastic, elongated material having an elongated length substantially longer than a width of the material; wherein the elastic bandage further comprises an engagement means joined to a first side surface of the elongated material, and being sized and adapted for the elongated material to be pulled around during use of the elastic bandage; wherein the elastic bandage further comprises a) a plate positioned on the second side surface of the elongated material, and opposite to the engagement means and/or b) a dome-shaped unit positioned on the second side surface of the elongated material, and opposite to the engagement means; and wherein the apex of the dome-shaped unit is projecting outwardly from the second side surface of the elongated material; and/or c) an object with one or more rounded edges positioned on the second side surface of the elongated material, and opposite to the engagement means; and wherein a rounded edge of said object is projecting outwardly from the second side surface of the elongated material.

An elastic bandage, sometimes referred to as a compression bandage, is stretchable and when properly used provides localized pressure and support. Elastic bandages are most commonly used after a part of the body, such as a leg, ankle, arm or wrist, is injured. For example, elastic bandages are used to provide support to an area of the body to prevent further injury, reduce or prevent swelling, maintain injured bones or a device (e.g., a splint) in proper position and other reasons.

The elastic bandage of the present invention is especially designed to address the issue of temporarily closing a wound in the axillary area and inguinal area. The engagement means works as a bandage wrapping direction changer when the elastic bandage is wrapped around a patient; and at the same time as a pressure point that ensures that the user can establish and maintain a constant high pressure directly on the injury even during movement of the casualty. The elastic bandage of the present invention can also be used as a conventional bandage, especially for use at places where conventional bandages tend to dislocate after being wrapped. The elastic bandage of the present invention can furthermore be used to keep pressure on and securing gauze or similar after packing a wound or large laceration. Additionally, the elastic bandage of the present invention can be used as a junctional pressure device, occluding blood flow to the extremities by pressuring directly on the iliac or axillary artery.

In one or more embodiments, the elastic bandage comprises a plurality of engagement means (such as 2-15), preferably grouped or closely positioned in relation to one another. Thereby the direction of the wrapping operation may be more easily changed, and the elastic bandage is furthermore less prone to dislocate when wrapped. Preferably, the elastic bandage comprises 3-5, e.g. 4, engagement means, preferably grouped, and preferably sharing the same base. The common base directs to pressure to a defined area, such as on a wound, of the patient.

The engagement means may be of different shapes, such as hook-shaped, mushroom-shaped, arrow-shaped (single bladed or multiple bladed, such as double bladed), spherically shaped, disc-shaped, or of mixed shapes thereof.

In one or more embodiments, the engagement means are shaped as a single bladed arrow, and where the tip of the arrow is pointing away from the centre of the base, shared by the plurality of engagement means.

In one or more embodiments, the engagement means comprises:
a base adapted for being connected to the first side surface of the elongated material;
a shank connected to the base and projecting outwardly from the first side surface of the elongated material, and adapted for the elongated material to be pulled around; and
a head portion connected to the shank, and adapted for preventing the elongated material from sliding of the shank.

The head portion may be of different shapes, such as hook-shaped, mushroom-shaped, arrow-shaped (single bladed or multiple bladed, such as double bladed), spherically shaped, disc-shaped, or of mixed shapes thereof.

In one or more embodiments, the elastic bandage comprises two or more engagement means (e.g. 2-15, such as 3-10, e.g. 4-7, and preferably 3-5), and wherein the two or more engagement means share a common base.

In one or more embodiments, the elastic bandage further comprises a plate positioned on the second side surface of the elongated material, and opposite to the engagement means. This configuration prevents the elastic bandage from twisting at the pressure site.

In one or more embodiments, the elastic bandage further comprises an object with rounded edges positioned on the second side surface of the elongated material, and opposite to the engagement means; and wherein a rounded edge of said object is projecting outwardly from the second side surface of the elongated material. Preferably, said object further comprises a flattened side-face adapted for facing the second side surface of the elongated material.

In one or more embodiments, the elastic bandage further comprises a dome-shaped unit positioned on the second side surface of the elongated material, and opposite to the engagement means; and wherein the apex of the dome-shaped unit is projecting outwardly from the second side surface of the elongated material. This configuration allows for a directed pressure on a defined area, such as on a wound, of the patient. It is important that the directed pressure is performed by an object with rounded edges, such as a dome-shaped object, to avoid damage to the tissue.

In one or more embodiments, the dome-shaped unit is attached to the second side surface of the elongated material.

In one or more embodiments, the dome-shaped unit is attached to the engagement means through a hole in the elongated material.

In one or more embodiments, the elastic bandage further comprises means adapted for moving the dome-shaped unit relative to the base of the engagement means. This configuration allows for adjusting the applied pressure on a defined area, such as on a wound, of the patient.

In one or more embodiments, the base and the dome-shaped unit each comprises a channel, and wherein the channels are aligned and adapted for receiving a fastening means.

In one or more embodiments, the base and the dome-shaped unit each comprises a threaded channel, and wherein the threaded channels are aligned and adapted for receiving a threaded fastening means (screw or bolt).

As used in the specification and the appended claims, the singular forms “a”, “an”, and “the” include plural referents unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

It should be noted that embodiments and features described in the context of one of the aspects of the present invention also apply to the other aspects of the invention.

### BRIEF DESCRIPTION OF THE FIGURES

FIG. 1 is a perspective view of an elastic bandage in accordance with various embodiments of the invention;

FIG. 2 is a close-up view of an engagement means in accordance with various embodiments of the invention;

FIG. 3 is a top view of an elastic bandage in accordance with various embodiments of the invention;

FIG. 4 is a perspective view of an elastic bandage in accordance with various embodiments of the invention, further comprising a plate positioned on the second side surface of the elongated material, and opposite to the engagement means; and

FIGS. 5-8 show an elastic bandage in accordance with various embodiments of the invention, further comprising a dome-shaped unit positioned on the second side surface of the elongated material.

### REFERENCES

100 Elastic bandage
200 Elongated material
202 First side surface
204 Second side surface
300 Engagement means
302 Base
304 Shank
306 Head portion
308 Mounting means
310 Threaded channel
400 Dome-shaped unit
410 Threaded channel
500 Means adapted for moving the dome-shaped unit
600 Plate

### DETAILED DESCRIPTION OF THE INVENTION

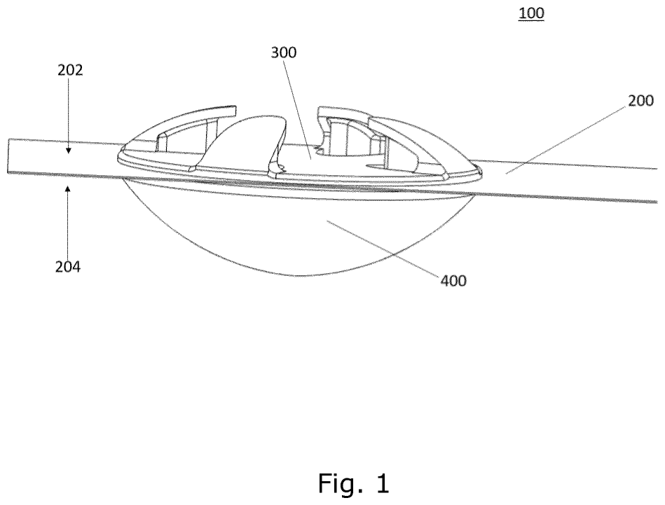

FIG. 1 is a perspective view of an elastic bandage 100 in accordance with various embodiments of the present invention. The elastic bandage 100 is shown comprising an elongated material 200 having an elongated length substantially longer than a width of the material. The elastic bandage 100 also comprises an engagement means 300 joined to a first side surface 202 of the elongated material 200, and sized and adapted for the elongated material 200 to be pulled around.

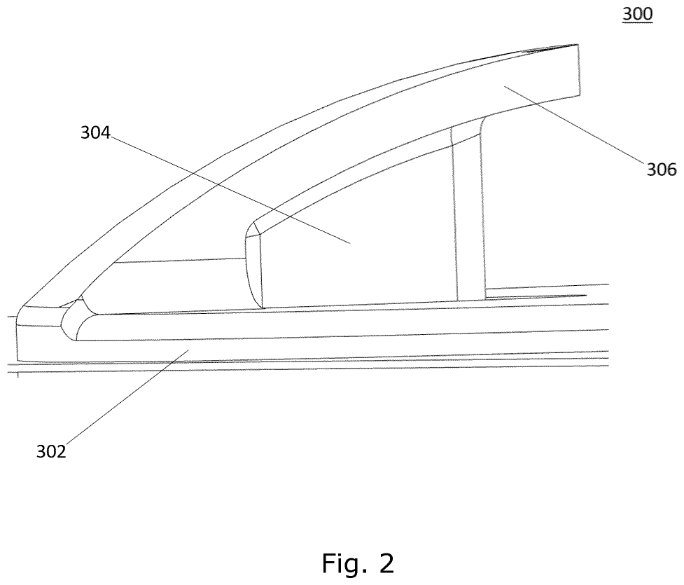

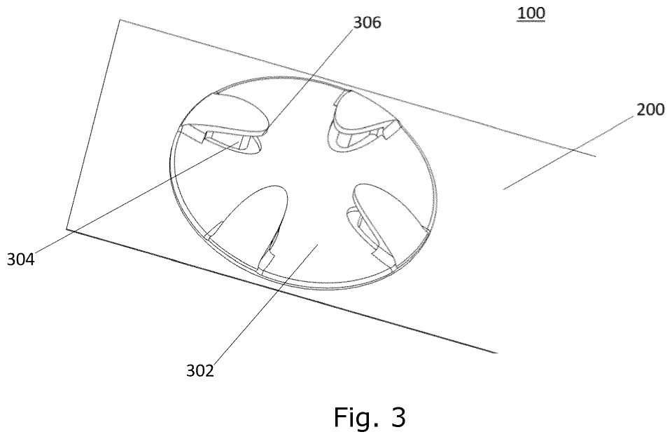

FIG. 2 shows a close-up view of the engagement means 300 in FIG. 1. The engagement means 300 comprises a) a base 302 adapted for being connected to the first side surface 202 of the elongated material 200; b) a shank 304 connected to the base 302 and projecting outwardly from the first side surface 202 of the elongated material 200, and adapted for the elongated material 200 to be pulled around; and c) a head portion 306 connected to the shank 304, and adapted for preventing the elongated material 200 from sliding of the shank 304. The engagement means 300 may be of different shapes, but this configuration has proven to be particularly resistant to the forces exerted thereon by the wrapped elongated material. The engagement means 300 is here shown shaped as a single bladed arrow, and where the tip of the arrow is pointing away from the centre of the base 302, shared by all four engagement means 300 (FIG. 3).

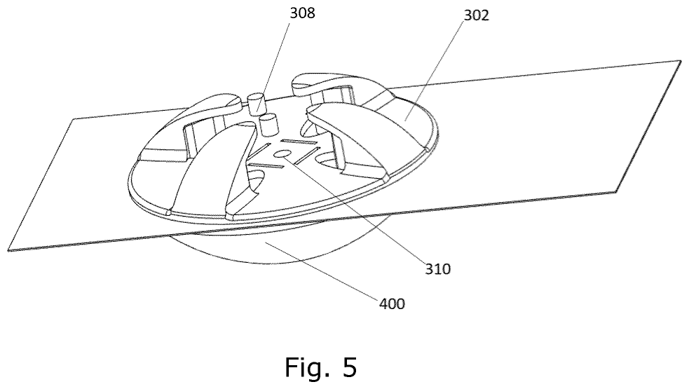

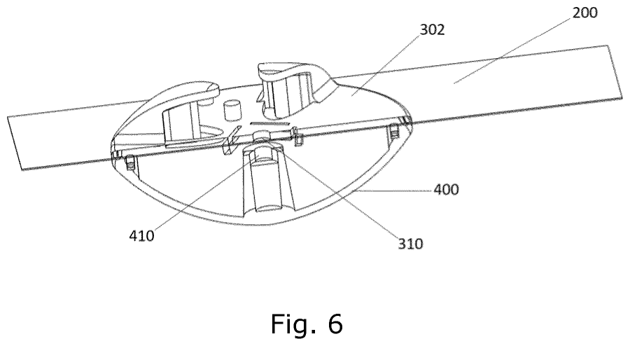

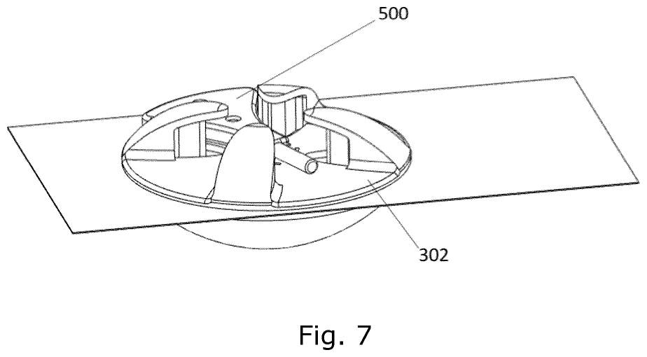

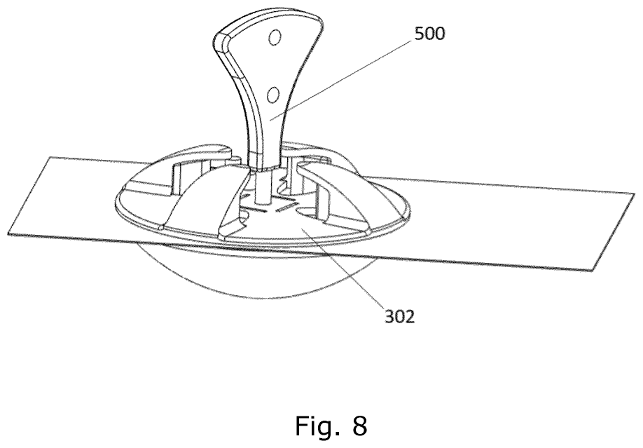

The elastic bandage 100 is also shown comprising a dome-shaped unit 400 (FIGS. 1, and 5-8) positioned on the second side surface 204 of the elongated material 200, and opposite to the engagement means 300. The apex of the dome-shaped unit 400 is projecting outwardly from the second side surface 204 of the elongated material 200. FIGS. 5-8 shows an embodiment, where the base 302 and the dome-shaped unit 400 each comprises a channel 310, 410 (FIG. 6), and wherein the channels 310, 410 (FIG. 6) are aligned and adapted for receiving a fastening means 500 (FIGS. 7-8). FIG. 5 shows a mounting means 308 adapted for mounting the fastening means 500 to the base 302 when not in use. In FIG. 7, the fastening means 500 is mounted to the base 302; and in FIG. 8, the fastening means 500 is positioned in the channels 310, 410.

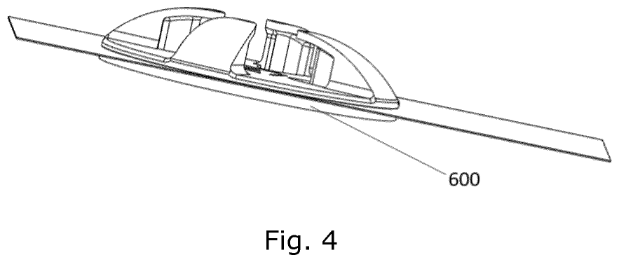

FIG. 4 shows an embodiment, where the elastic bandage comprises a plate 600 positioned on the second side surface 204 of the elongated material 200, and opposite to the engagement means 300. This configuration prevents the elastic bandage from twisting at the pressure site.

1. An elastic bandage, comprising:
an elastic, elongated material having an elongated length substantially longer than a width of the material;
an engagement means joined to a first side surface of the elongated material, and being sized and adapted for the elongated material to be pulled around during use of the elastic bandage;
wherein the elastic bandage further comprises a dome-shaped unit positioned on the second side surface of the elongated material, and opposite to the engagement means; and
wherein the apex of the dome-shaped unit is projecting outwardly from the second side surface of the elongated material.

2. The elastic bandage according to claim 1, wherein the elastic bandage comprises three or more engagement means, and wherein the three or more engagement means share a common base.

3-5. (canceled)

6. The elastic bandage according claim 1, wherein the engagement means comprises:
a base adapted for being connected to the first side surface of the elongated material;
three or more shanks connected to the base and projecting outwardly from the first side surface of the elongated material, and adapted for the elongated material to be pulled around during use of the elastic bandage; and
a head portion connected to each shank, and adapted for preventing the elongated material from sliding of a shank when pulled around during use of the elastic bandage.

7. (canceled)

8. The elastic bandage according to claim 1, wherein the dome-shaped unit is attached to the second side surface of the elongated material.

9. The elastic bandage according to claim 1, wherein the dome-shaped unit is attached to the engagement means through a hole in the elongated material.

10. The elastic bandage according to claim 9, further comprising means adapted for moving the dome-shaped unit relative to the base of the engagement means.

11. The elastic bandage according to claim 1, wherein the base and the dome-shaped unit each comprises a channel, and wherein the channels are aligned and adapted for receiving a fastening means.

12. The elastic bandage according to claim 1, wherein the dome-shaped unit is generally non-deformable during intended use.

13. The elastic bandage according to claim 1, wherein each engagement means has any one of the following shapes: hook-shaped, mushroom-shaped, arrow shaped, spherically shaped, disc-shaped, or of mixed shapes thereof.

14. The elastic bandage according to claim 1, wherein each engagement means is shaped as a single bladed arrow, and where the tip of the arrow is pointing away from the centre of the base, shared by the plurality of engagement means.
